(19) United States
(12) Patent Application Publication
(10) Pub. No.: US 2024/0066430 A1
(43) Pub. Date: Feb. 29, 2024

(54) APPARATUS, SYSTEM AND METHODS FOR PROCESSING OF HARVESTED CANNABIS PLANT MATERIAL

(71) Applicant: EQALIS GROUP NEW ZEALAND LIMITED, Hamilton (NZ)

(72) Inventor: Greg Charles MISSON, Hamilton (NZ)

(21) Appl. No.: 18/259,095

(22) PCT Filed: Dec. 24, 2021
(86) PCT No.: PCT/NZ2021/050233

§ 371 Date: Jun. 23, 2023

(30) Foreign Application Priority Data
Dec. 24, 2020 (NZ) 771591
Dec. 24, 2020 (NZ) 771593
Mar. 30, 2021 (NZ) 774531
Dec. 23, 2021 (NZ) 783924

(51) Int. Cl. B01D 11/02

(52) U.S. Cl. CPC B01D 11/0288; B01D 11/0207; B01D 11/028

(57) ABSTRACT

The present technology is directed towards providing an apparatus, system and method for the cost-effective extraction of compounds of interest, such as CBD and THC, from freshly harvested cannabis plant material. The system includes multiple processing modules, each having an agitator, tank and filter basket and is communicative with a separator. The tanks are filled with a cooling agent and the filter baskets provided with cannabis plant material which is immersed and agitated in each tank. The filtered outflow from each tank is a mixture of cooling agent and cannabis plant material that is transferred to a separator to extract the compounds of interest. The filtered outflow from each processing mod-ule is timed to provide a continuous flow rate to the separator.

### CROSS-REFERENCE TO RELATED APPLICATIONS

This application is a national phase application under 35 U.S.C. § 371 of International Application No. PCT/NZ2021/050233 filed Dec. 24, 2021 entitled, “APPARATUS, SYSTEM AND METHODS FOR PROCESSING OF HARVESTED CANNABIS PLANT MATERIAL,” which claims the benefit of and priority to New Zealand Patent Application No. 771591 filed Dec. 24 2020; New Zealand Patent Application No. 771593 filed 24 Dec. 2020; New Zealand Patent Application No. 774531 filed Mar. 30, 2021; and New Zealand Patent Application No. 783924 filed Dec. 23, 2021, the contents of each of which being incorporated by reference in their entireties herein.

### TECHNICAL FIELD

The present disclosure relates to an apparatus, system and method for the processing of harvested cannabis plant material. The embodiments of the present disclosure have particular application to the extraction of target compounds cannabinoids, such as cannabidiol or tetrahydrocannabinol and Varian analogues, from freshly harvested cannabis.

### BACKGROUND

Cannabis, primarily Cannabis sativa but also Cannabis indica and Cannabis ruderalis, for medicinal purposes such as pain relief, is legally recognised in many countries, including the United States and New Zealand. It can be provided to the consumer as a dried product (leaves or flowers) or in as a dosage in a conventional pharmaceutical form, such as a pill, tablet, or as an oil/liquid for oral consumption. There is also an increasing need to separate specific compounds, in specific forms, from cannabis plant material for use in medicaments for treatment of specific health conditions.

Consequently, cannabis plants are being cultivated on an increasing scale to meet demand. However, cultivating cannabis plants on a commercial scale has a number of issues due to space constraints, availability of resources, and technological inefficiencies. This means the cost of purchasing medicinal cannabis can be significant, a reflection of the expense involved in growing, processing, and extracting from the cannabis plant the valuable components, the cannabinoids, such as cannabidiol (CBD) or tetrahydrocannabinol (THC), into a form that is vendible to the consumer.

This expense can make it difficult for persons requiring medicinal cannabis to access the product. It can also make it prohibitively expensive for potential cannabis farmers to enter the market, which in turn keeps the price of cannabinoid containing medicines high.

To optimise growth of the cannabis plant, they are often grown indoors and most growers supplying medicinal cannabis to the market take this approach. While this allows the grower to better control the temperature, light conditions, and security of the plant crop, a drawback is that a major amount of infrastructure is required, from growing space, i.e. buildings, to electrical equipment, and watering equipment.

Once harvested, the cannabis plant material degrades relatively quickly and is vulnerable to mold and pestilent insects. The harvested cannabis plant material also has a significant moisture content, which can be as much as 70%, which contributes to its undesirable breakdown. This moisture needs to be eliminated or at the very least significantly reduced before the plant material can be further processed. The drying process is both labor and energy demanding, as the plants need to be trimmed and stored in a relatively dehumidified environment for drying for up to two to three weeks.

Harvesting of the cannabis plants usually involves removal of a major proportion of their biomass, the stems and fan leaves, leaving only the sugar leaves and flowers. These contain the valuable component of interest, a structure known as a trichome, a bulbous resinous gland on a thin stem. It is the trichomes that contain the target compounds, cannabinoids such as CBD and THC, that need to be extracted with further processing.

A common commercial approach to extracting target compounds/valuable components from cannabis plant material involves a multi-stage process. First, the moisture level in the plant matter containing the trichomes is reduced to only around 10% moisture. The cannabinoids of value can then be extracted, which usually entails the application of solvents, such as ethanol, to the trichomes. However, this requires the use of complex scientific equipment, with skilled staff being necessary to operate the processing machinery and who need to be remunerated appropriately. The solvents used in the processing can be hazardous and must be stored in safe and secure environments, and also disposed of appropriately.

Another approach to separating out the trichomes from the plant matter involves immersing it in a tank of cool or chilled water and gently agitating the resulting mixture. This separates the trichomes from the rest of the plant matter and they can then be captured and extracted from the water using a separator. An example of such an arrangement is disclosed in PCT Application No. PCT/CA2018/051197. However, a drawback to this arrangement is that it can be very wasteful of water, particularly on a commercial scale, as it typically requires 20 parts of water to one part plant matter for optimum operation. After the separation of the trichomes from the water, it is then discarded as waste. Furthermore, the separation of the trichomes from the plant matter can also be relatively prolonged and time inefficient.

As will be appreciated there is considerable capital and operational expense in growing and processing cannabis plants for the purpose of supplying medicinal cannabis. Together with the time and processing inefficiencies of existing extraction processes, this has a bearing on the retail price for the end product. In some markets, this may mean medical cannabis or medicines containing cannabinoids may be financially inaccessible to at least some of those individuals who need it for treatment of health problems and/or pain relief.

A solution to the aforementioned problems is desired.

### BRIEF SUMMARY

The present disclosure is directed towards providing an apparatus, system, and method for the cost-effective extraction of compounds of interest, e.g. value components, from freshly harvested cannabis plant material. These compounds of interest include, but are not limited to, CBD and THC, and Varian analogues thereof.

In particular, the present technology provides an apparatus, system, and method for the removal of value components of the cannabis plant material, the trichomes. These are the parts of the plant that are particularly high in CBD and THC compounds at certain stages of the life of the plant.

According to one aspect of the present technology, there is a provided a method of separating trichomes from harvested cannabis plant material, wherein the method includes the steps of:
a) charging a first vessel with a quantity of harvested cannabis plant material and a quantity of a cooling agent to create a first mixture;
b) agitating the first mixture in the first vessel to produce a first laden quantity of filtrate containing at least some trichomes and a first waste quantity of cannabis plant material;
c) withdrawing the first laden quantity of filtrate from the first vessel and transferring to a separator;
d) operating the separator to remove at least a portion of the trichomes from the first laden quantity of filtrate;
e) charging a second vessel with a quantity of harvested cannabis plant material and a quantity of cooling agent to create a second mixture;
f) agitating the second mixture in the second vessel to produce a second laden quantity of filtrate containing at least some trichomes and a second waste quantity of cannabis plant material;
g) withdrawing the second laden quantity of filtrate from the second vessel and transferring to the separator;
h) operating the separator to remove at least a portion of trichomes from the second laden quantity of filtrate;
   wherein steps e) to g) are timed to provide a substantially continuous outflow of filtrate from the vessels.

According to another aspect of the present technology, there is provided a method as substantially described above herein, but including a further step of:

i) returning at least a portion of the first laden quantity of the filtrate to the first vessel after step d).

According to another aspect of the present technology, there is provided a method substantially as described above herein, but further including:
j) removing the first waste quantity of cannabis plant material from the first vessel during or after step c)
k) cleaning the first vessel; and
l) charging the first vessel with a quantity of harvested cannabis plant material and a quantity of a cooling agent to create a third mixture.

Optionally, the cooling agent is a slurry of ice and water. Alternatively, the cooling agent is chilled water.

Optionally the cooling agent is chilled to a temperature of between substantially 0° Celsius to substantially 5° Celsius. Even more Optionally, the cooling agent is chilled to a temperature of substantially 0° Celsius.

Optionally, the ratio of cooling agent to plant material is from between substantially 0.5:1 to substantially 10:1. Even more Optionally, the ratio of cooling agent to plant material is between substantially 1:1 to 5:1. In a particularly preferred embodiment, the ratio of cooling agent to plant material is approximately 2:1.

Optionally, the agitator mechanically works the plant material into a slurry with the cooling agent.

Optionally, a substantially continuous outflow of filtrate from the vessels is to a separator. In some examples, the substantially continuous outflow of filtrate from the vessels may be to a holding tank.

Optionally, step a) is performed over a period of 15 to 45 minutes. Even more Optionally, step a) is performed over a period of approximately 30 minutes.

Optionally, step b) is performed for between 30 minutes to six hours. Even more Optionally, step b) is performed for approximately 90 minutes.

Optionally, steps c) and d) are performed concurrently with step b).

Optionally, step e) is performed at least partially concurrently with one or more of steps b) to d).

Optionally, step i) is performed for between 5 and 20 minutes. Even more Optionally, step i) is performed for between 10 to 15 minutes.

Optionally, steps a) to d) are performed for between one hour to eight hours. Even more,

Optionally, steps a) to d) are performed for approximately 150 minutes.

Optionally, step k) includes recycling a cleaning agent through the vessel.

Optionally, in step k), the cleaning agent is water and/or mild detergent.

Optionally, prior to step a), the harvested cannabis plant material undergoes a pre-processing step. In a preferred embodiment, in the pre-processing operation, the stalks and/or fan leaves are substantially removed or reduced in size, e.g., through the use of cutting blades or the like. In an alternative embodiment, the harvested cannabis plant material is passed through a cutting and/or milling machine.

Optionally, during the pre-processing operation, the harvested cannabis plant material is chilled and/or de-humidified. In a preferred embodiment, the chilling agent is chilled air or water.

According to a further aspect of the present technology, there is provided a system for use in the method of processing harvested cannabis substantially as described above, wherein the system includes:
a separator; and
a plurality of vessels, wherein each vessel is configured to receive a cooling agent and harvested cannabis plant material and wherein each vessel is provided with:
   a charger for harvested cannabis plant material;
   a charger for a cooling agent;
   an agitator; and
   an outlet that is communicative with the separator.

Optionally, the vessel is a tank. Even more Optionally, the vessel is a substantially cylindrical open tank.

Optionally, the tank has a continuous side wall and a base.

Optionally, the outlet of the tank is at or proximate the base.

Optionally, the tank includes a filtering medium.

Optionally, the filtering medium is a substantially cylindrical basket.

Optionally, the dimensions of the cylindrical basket are 80% to 95% of the dimensions of the tank.

Optionally, the cylindrical basket includes a continuous side wall with a plurality of perforations. Even more Optionally, the continuous side wall is fabricated as a mesh.

Optionally, the agitator is configured to mechanically work and break up the harvested cannabis plant material into shreds and/or particulate matter and work it into a slurry with the cooling agent.

Optionally, the agitator includes a plurality of blades and tines to mechanically work and break up the harvested cannabis plant material into shreds and/or particulate matter.

Optionally, the vessels are communicative with the separator. Even more Optionally, the system includes a flow path from the outlet of each vessel to the separator. In a particularly preferred embodiment, the flow path is a pipe line between the outlet and the separator.

Optionally, the system includes a first pipeline from the separator back to one or more of the vessels.

Optionally, the system includes a second pipeline from the separator to a processing station.

### BRIEF DESCRIPTION OF DRAWINGS

One of more embodiments of the present technology will be described below by of example only, and without intending to be limited, with reference to the following drawings, in which like reference numerals refer to similar elements including:

FIG. 1 is a flow chart processing harvested cannabis;

FIG. 2 is a schematic of a system for processing harvested cannabis in accordance with one example of the present technology;

FIG. 3 shows a further schematic of the system of FIG. 2;

FIG. 4 shows a schematic of an example of a plurality of systems configured to process harvested cannabis plant material;

FIG. 5 shows a schematic of an alternative example of a plurality of systems configured to process harvested cannabis in parallel; and

FIG. 6 shows a graphical representation of the productivity of an example of a plurality of systems operated in accordance with one example of the present technology.

### DETAILED DESCRIPTION

1.1. Processing Method for Freshly Harvested Cannabis Plant Material

Embodiments of the present technology relate to methods and equipment used to process harvested cannabis.

Harvested cannabis should be understood to mean cannabis plant material that has reached an age appropriate for processing, which is typically eight to ten weeks after flowering. The cannabis plant comprises three main parts; the stalk, the leaves (fan leaves, which are the larger, sun-collecting leaves of the plant, and the sugar leaves, that form around the flower, or bud, of the plant), and the flower.

The value components of the cannabis plant are substantially contained in the trichomes. These are found primarily in the sugar leaves and the female flowers of the plant. The trichomes are not found in major quantities in the male flowers.

The colour of the trichomes may be indicative of when the plant should be harvested; if clear, there is minimal THC and/or CBD present, if somewhat translucent or cloudy an increasing amount of THC and/or CBD is present. If amber, the THC and/or CBD concentration declines. The grower will optionally harvest the cannabis plants when the THC and/or CBD presence in the trichomes is optimal.

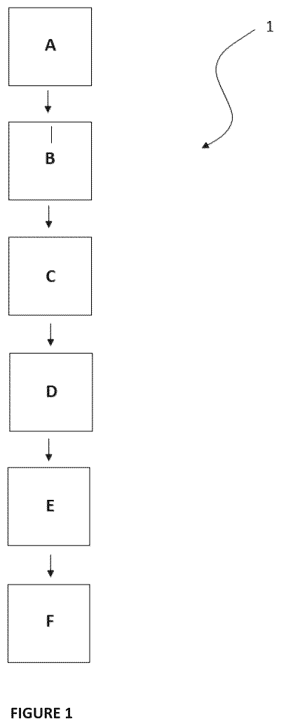

One method of harvesting essentially involves separating the stalk from the root structure using cutting equipment. Following harvesting, the resulting plant material goes through several steps of processing, generally indicated as arrow 1, as indicated in FIG. 1: A) pre-processing; B) pre-concentration; C) clarification; D) de-watering; E) extraction; and F) refining.

Each of these steps shall now be described.

5.1.1 Pre-Processing

Once harvested, the cannabis plant material undergoes an initial pre-processing step A, which involves the removal of the larger, lower value/non-productive components of the plant, such as the stalks and fan leaves. This leaves the valuable components of the plant material, the buds/flower and sugar leaves for further processing in accordance with the present technology.

In one example, the pre-processing step involves the de-stalking of the plant material. The stalks of the cannabis plant are of comparatively low value. Alternatively, the value components of the plant material may be harvested in the growing area, leaving the stalks and/or fan leaves behind.

In one example, the pre-processing step involves introducing the plant material to a cutting and/or milling machine. Optionally, during this process, the plant material is reduced to dimensions of around 1 mm to 50 mm. These dimensions may be dependent on the technology involved in following steps and the genetic traits of the specific plants being processed. For example, in some instances, the cutting and/or milling may reduce the dimensions of the plant material to around 1 mm to 25 mm.

In some examples of the present technology, the plant material may be cooled and/or de-humidified for a short period of time during or immediately following the pre-processing step A. In one example, the cooling may be achieved through the introduction of chilled water to the cutting and/or milling machine concurrently with the plant material. In another example, chilled air may be directed onto the plant material as it is conveyed to or passes through the cutting and/or milling machine.

5.1.2 Pre-Concentration

Following pre-processing, the remaining plant material, the flowers and sugar leaves, are concentrated and then further separated into a filtrate stream and a waste stream in a pre-concentration step B.

The filtrate stream should be understood to contain the overwhelming majority of the value components, i.e. the cannabinoids, while the waste stream should be understood to be the low value-by product resulting from the pre-concentration step. For example, the waste stream comprises the larger particulate plant matter, such as leaves and residual stalks and stems. This waste is removed from processing at the completion of the pre-concentration step.

An important aspect of the pre-concentration step B is the introduction of the pre-processed cannabis plant material to a quantity of cooling agent and then agitating the mixture. This agitation is particularly aggressive and mechanically works the immersed cannabis plant material. This helps to expeditiously separate the value components, particularly the trichomes, from the plant material. It may also help to break up the trichomes into smaller fragments and components.

The term cooling agent should be understood as meaning a fluid (e.g. a liquid or a gas) which reduces the temperature of the plant material when they come into contact with each other. In one form, the cooling agent is a slurry of chilled water and particulate ice, in the form of cubes or chunks, that has been reduced in temperature to substantially to 0° Celsius, or close to 0° Celsius. This is advantageous for when trichomes are exposed to temperatures close to freezing, they become brittle and are more easily separated from the flowers and leaves with appropriate agitation action.

In another example, the cooling agent may be brine (salt water) with particulate ice. This may be useful since brine can be used in following steps for rinsing the filtrate. Its use as a cooling agent could optimise the efficiency of the processing of the filtrate. It also allows for the further reduction of the temperature of the cooling agent since brine has a lower freezing point than fresh water.

In further examples, the cooling agent may include additives for the purpose of improving efficiencies in the processing of the filtrate. In one such example, a pH adjuster, such as salt, may be introduced to the slurry of ice and water. In another such example, a surfactant may be added to the cooling agent; this may assist in the separation of the trichomes from the remaining plant matter and/or cooling agent.

This reduction in temperature increases the efficiency of the pre-concentration phase and the separation of the plant material being processed into filtrate and waste streams. In one example, the filtrate contains around 10% or less of the plant material that is pre-concentrated, with the remaining 90% or so forming the waste.

The use of a cooling agent to reduce the temperature of the harvested plant material increases the efficiency of the pre-concentration phase and the resultant separation of the plant material into filtrate and waste streams. In one example, the filtrate contains around 10% or less of the plant material, much of which is the trichomes, that is pre-concentrated, with the remaining 90% or so forming the waste stream.

The quantity of cooling agent used in the pre-concentration step B may depend on the type used and the amount of plant material to be processed at any one time. In one form, when the cooling agent is a slurry of chilled water and ice, the ratio of cooling agent to plant material is from 0.5:1 to 10:1 or, more notably, 1:1 to 5:1. In a particularly preferred form, the ratio of cooling agent to plant material is approximately 2:1.

The present technology relates to a system, apparatus and method of using the same to perform the pre-concentration step, and specifically the removal of certain parts of harvested cannabis plant material, the trichomes, and processing these into a filtrate stream and a waste stream. It should be appreciated that in some examples, the waste may still contain some trichomes; in these examples, at least a portion of the waste stream may be recirculated back into the system and the method repeated to extract at least some of these trichomes. This is an important aspect of the present disclosure as it helps make the overall processing of harvested cannabis plant material more efficient than conventional systems, while optimising the use of the cooling agent, thereby minimising wastage. This provides a method and system for processing of harvested cannabis at volumes that is more economically viable on a commercial scale of operation.

5.1.3 Clarification

The clarification step C further separates the filtrate stream by removing any remaining large particulate plant material of low value.

This may involve further fine filtering of the filtrate. Alternatively, the clarification step may involve recycling the filtrate for a period of time through the pre-concentration step previously discussed.

5.1.4 De-Watering

After the clarification step, the filtrate is predominately a liquid containing the remnants of the trichomes and a significant water content. The de-watering step D involves the use of conventional filtration techniques as should be known to the skilled addressee and dehumidification to reduce the moisture content and dry the filtrate and leave behind a solid material.

5.1.5 Extraction

To extract target compounds, e.g., cannabinoids from the dried filtrate, known extraction techniques may be employed by the skilled addressee in an extract step E. In one example, a solvent such as ethanol may be used, in combination with filtration techniques.

5.1.6 Refining

The extract of the previous step may be further processed in a refining step F using conventional distillation and/or crystallisation/chromatography techniques that will be readily understood by the skilled addressee.

1.2. System

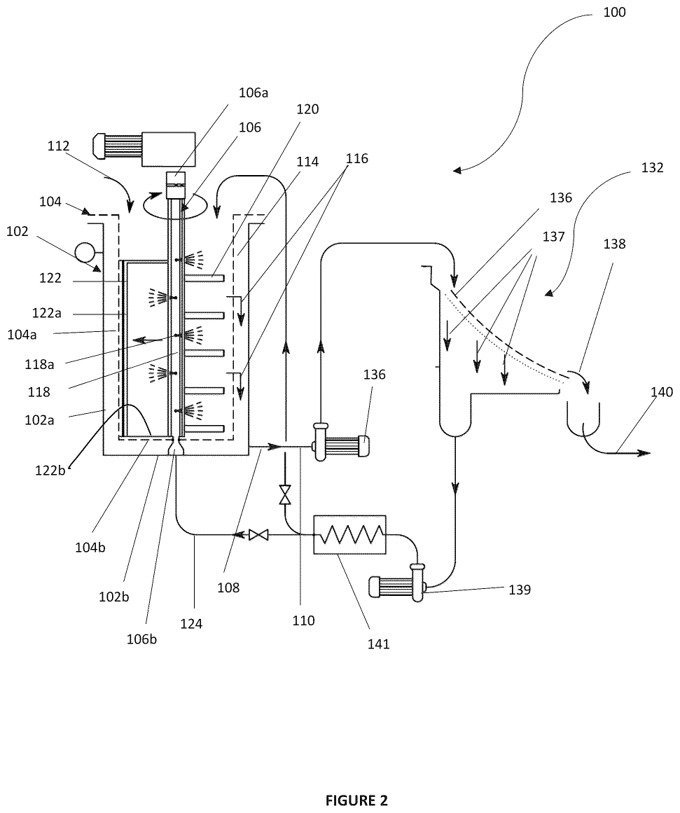

In one example illustrated in FIG. 2, the system (generally indicated by 100) of the present technology includes a vessel 102, each provided with a filtering medium in the form of a basket 104, and an agitator 106 disposed within the basket. In use, this serves to stir and vigorously agitate the mixture of cannabis plant material and cooling agent when introduced to the vessel, as will be discussed below. However, other examples of the system may employ other means of agitating the mixture; for example, a static mixer (not shown) may be employed downstream of the vessel. Alternatively, a pump (not shown) may be used for this purpose. Furthermore, in some examples, the basket may be omitted and instead a filtering screen may be provided at or proximate the outlet 108 of the vessel to prevent larger particulate cannabis plant material (which may be stalks or the like and thus forming biomass that may be of low value) from exiting the vessel.

The agitation is another aspect of the present disclosure. Aggressive agitation, compared to the more gentle methods used in the related art, releases more trichomes into the cooling agent. This potentially optimises the extraction of the value components of the cannabis plant material.

The vessel, basket and, if present, the agitator are arranged and configured to best optimise the implementation of the pre-concentration step of the processing method.

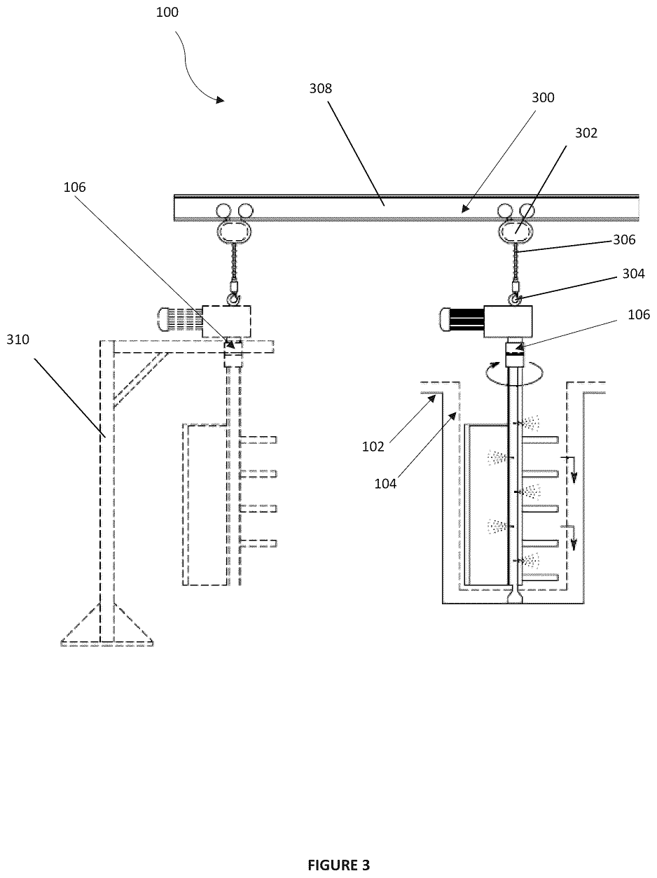

In some examples, the system 100 may also include a transfer mechanism (not shown in FIG. 2 and but identified as 300 in FIG. 3) that places and removes the baskets 104 and/or agitators 106 into and out of the vessels 102 as required.

The respective essential and optional parts of the system used to perform the method shall now be described in detail.

1.2.1. Vessel

Each vessel 102 should be understood to be a container that in use receives the plant material being processed and a quantity of cooling agent. In this example, the separation of the plant material into filtrate, a slurry of plant material and cooling agent, and waste streams takes place within the vessel. However, in some examples, the separation of the filtrate may take place downstream of the vessel.

In one example of the present technology, the vessel 102 is an open tank, with at least its internal surface configured as a cylinder and thus having substantially a circular cross-section. However, this is not meant to be limiting and other shapes and profiles for the vessel will be readily envisaged by persons skilled in the art. The vessel shall now be referred to as a tank throughout the remainder of the present specification.

The 102 tank has a continuous side wall 102a and a base 102a and is fabricated from an appropriate material, such as stainless steel or the like. In use, the tank receives the basket 104; the tank may already be pre-filled with the cooling agent prior to the introduction of the basket or alternatively, the cooling agent may be introduced afterwards.

In an example, the tank 102 has a diameter of approximately 2 metres and has a height that is at least twice its diameter. In this configuration the tank has a volume of approximately at least 4,000 litres. However, it will be appreciated by persons skilled in the art that the dimensions of the tank may be scaled up or down depending on the intended volume of harvested cannabis plant material that is intended to be processed at any one time.

The tank 102 is provided with an outlet 108 as seen in FIG. 2, which in one example is located or otherwise positioned at or proximate the base 102b. This allows liquids received by the tank to be drained away for further processing as required. It will be appreciated that at least some of the drained liquid is the filtrate, containing the trichomes and their cannabinoids of interest as suspended particulate matter within the filtrate.

The base 102b of the tank 102 may be flat, with the outlet 108 positioned centrally or alternatively as shown in FIG. 2, at a lower edge of the side wall 102a. In other examples, the base of the tank may be in an inverted conical form, with the outlet positioned at the lower most point; this configuration may help with efficient drainage of the liquid contents of the tank.

The outlet is communicative with a discharge pipe 110. This may be under pressure to better optimise flow of the filtrate through the pipes. In some examples, the outlet may include a screen to capture any larger particulate matter and preventing it entering the filtrate.

In some examples, the tank 102 is configured with a supply line or charger (not shown) for the introduction of the cooling agent. This may be to the sides of the tank or alternatively, a separate structure entering the open top of the tank.

1.2.2. Filtering Medium

In one example, the filtering medium should be understood to be a container that is configured to act as a filter. In one example, the filtering medium is a basket 104 and shall be referred to as such throughout the present specification.

In use, the harvested and pre-processed cannabis plant material is placed 112 in the basket 104. This may be by hand or alternatively may be through the use of a charger or hopper or the like (not shown) as will be readily apparent to a skilled addressee.

It will be appreciated that in preferred examples of the present technology, the basket 104 is configured to be substantially complementary to the tank 102 of the system 100. In a particular example of the present technology, the basket is a cylindrical structure having a continuous side wall 104a and a base 104b. It will be appreciated that in this example, the tank that receives the basket is similarly a cylindrical structure.

In one example, the dimensions of the basket 104 are such that it is 5% to 20% smaller than the tank 102 in which it is to be placed. This means that when centrally located within the tank, there is clearance between the continuous wall and base of the basket and that of the tank.

In one example, the basket may be between 1.9 to 1.4 metres in diameter and 3.8 to 3.2 metres in height. However, it will be appreciated by persons skilled in the art that, as previously noted in respect of the tank, the dimensions of the basket may be scaled up or down depending on the intended volume of cannabis plant material that is intended to be processed at any one time.

In some examples, the base 104b of the basket 104 may be configured with structures that are complementary to structures provided to the upper surface of the base 102b of the tank 102. This helps to anchor the basket relative to the tank, particularly when it is being agitated.

In one example of the present technology, the basket 104 is fabricated from stainless steel sheet having perforations of approximately 0.3 mm to 3 mm. In other examples, the walls 104a and base 104b of the basket are fabricated from stainless steel mesh.

Once filled with the plant material to be processed, the basket 104 is transferred to the tank 102 for the pre-concentration phase. Alternatively, the basket is placed within the tank and freshly harvested and pre-processed cannabis plant material is then introduced to the basket. The tank may already contain the cooling agent prior to the basket being placed therein or alternatively the cooling agent is introduced to the tank after the basket.

It will be appreciated that, in use and following the introduction of the cooling agent, the basket 104 serves as a filter for the plant material being processed. The larger particulate matter forms the waste material that remains in the basket while smaller particulate matter and dissolved compounds, including the parts of particular value, the trichomes, forms the filtrate 116 which passes through the walls 104a of the basket and is allowed to exit the tank 102 via the outlet 108 along with the cooling agent. The filtrate is largely a slurry of shredded plant material, broken up into particulate matter through the action of the agitator, and cooling agent.

In one example, the system 100 includes at least two baskets. This is advantageous for, while one basket is being processed in accordance with the present method, the second basket can be prepared for use by charging it with harvested cannabis plant material in anticipation of being processed. Then, while the first basket is being cleaned and recharged with plant material, the second basket can be processed.

1.2.3. Agitator

In one example of the present technology, the system 100 includes an agitator 106. In use, the agitator is configured to agitate and thereby mix the cooling agent and harvested cannabis plant material in the filter medium 104.

As discussed above, the tank 102 may be filled with a cooling agent. The action of the agitator serves to provide the necessary forces to encourage physical breakdown of the plant material. In particular, the agitator encourages separation of the now significantly cooled trichomes from the remaining plant material so that they become entrained in the cooling agent as suspended particulate matter. This is an important aspect of the present technology.

Referring to FIG. 2, in one example, the agitator 106 comprises an elongate, vertically orientated shaft 118. A plurality of agitating blades or tines 120 extends substantially perpendicularly from the shaft at regular intervals.

The agitator 106 is provided with a drive motor 106a to rotate the agitator about a vertical axis. This action, with the blades 120, serves to mix and agitate the immersed plant material and encourage the separation of same into the low value waste and the high value filtrate.

In examples, the motor 106a acts to rotate the agitator at speeds ranging from 20 to 200 revolutions per minute or more. The higher speeds are preferred for aggressive agitation and breakdown of the immersed plant material to release the trichomes containing the value components. It can also help in breaking down of the trichomes into fragments.

In some further examples, the motor may be set to provide, at regular intervals, a pulsing action where the speed of rotation is increased for a period of time. Alternatively, the motor may be operative such that for a period of time, the agitator rotates in a first direction, and then a second direction. In both of these examples, this enhances the effect of the agitator upon the plant matter.

In some further examples not shown here, to supplement the effect of the agitator, the blades may be configured with tines or the like extending in various orientations to further thresh the plant material in various directions when the agitation is in motion.

In the illustrated example, the base 106b of the agitator 106 may be configured to key into or otherwise engage with the basket 104, optionally at the base 104b of the basket. In preferred examples, this may be by way of a bearing hub or the like. This helps ensure the structural integrity and minimise stresses during operation of the agitator.

In one example, at least a portion of the shaft 118 of the agitator 106 is hollow, with a series of apertures 118a allowing communication between the interior of the shaft to the exterior, arranged at regular intervals along its length. In this example, the agitator is provided with a pipe 124 for pressurised chilled water which is directed through the hollow tube of the shaft and out the apertures provided along same. In some examples, the apertures may be configured with nozzles or similar structures to help optimise the desired radial flow path.

In use, the pressurised water that is directed through the shaft 118 of the agitator 106 and emitted out the apertures 118a serves two purposes. Firstly, depending on the extent of the pressure at which it exits the shaft of the agitator, it provides some impact forces on the plant material, supplementing the action of the agitator and assisting in the separation of trichomes from the plant material. Secondly, it creates a radial flow path within the basket and tank, encouraging further separation the finer particulate plant matter, including the trichomes, from the plant material being processed and directing towards the space 114 between the basket 104 and the inner surfaces of the tank 102.

In some examples, the cooling agent may be introduced to the tank via the hollow shaft 118 of the agitator 106 as shown in FIG. 2 but in other examples, as previously discussed, the cooling agent may be delivered to the tank via a separate charger or supply line.

In a further example, the agitator 106 may include a scarper 122 which, in use, bears against the continuous wall 104a of the basket 104 and scrapes its surface. In one form, the scarper is an elongate bar 122a mounted to a pair of arms 122b extending perpendicularly from the shaft of the agitator; as the agitator rotates, so does the scarper. This helps optimise the filtering of the plant material performed by the continuous wall and/or base of the basket, minimising any blockages that may occur.

In some examples, the scarper 122 may be configured to receive a plastic insert (not visible) which is the scarping surface that contacts the continuous wall 104a of the basket 104 in use. In this form, the use of the plastic insert reduces the risk of damage to the walls of the basket.

In some examples, the scarper 122 may be arranged to encourage the development of the radial flow path that directs the filtrate 116 towards the space 114 between the basket 104 and tank 102.

1.2.4. Transfer Mechanism for Basket and/or Agitator

In one example of the present technology as shown in FIG. 3, the system 100 is provided with a transfer mechanism 300 to facilitate the placement of the basket 104 and/or agitator 106 within the tank 102. On the left in dashed lines is an agitator in a stored position and on the right an agitator shown in an “in use” position, where it is disposed within the tank.

In its simplest form, the transfer mechanism 300 includes lifting equipment in the form of a winch 302 provided with a hook 304 and cable 306 which engage with complementary fittings (not visible) provided to the upper surfaces of the agitator 106 and baskets 104. In alternative examples not shown, the cable may be provided with a loop which engages with hooks or similar fittings provided to the agitator and baskets. The skilled addressee should readily envisage and implement a transfer mechanism suitable for use with the present system.

In some examples, the transfer mechanism may include a gantry 308 along which the lifting equipment 302 is able move. This allows the basket 104 and/or agitator 106 to be moved to a location clear of the tank 102.

In some examples, the agitator 106 may be provided with a frame or support stand 310 for when it is not in use. When the agitator is located within the frame, the transfer mechanism 300 may be used to place the basket 104 in the tank 102 and/or remove it from same.

1.2.5. Separator

In one example of the present technology as shown in FIG. 2, the system 100 includes a separator 132 which will be understood to be downstream of the tank 102 and receives laden quantities of filtrate 116 via the outlet 108 and discharge pipe 110 of each tank. The separator will be understood to be an apparatus that processes the filtrate into a high value stream and a low value stream; the former includes the trichomes of interest while the latter may be spent or partially spent cannabis plant material from which at least some of the trichomes have been removed.

The discharge pipe 110, pressurised by a pump 134, directs the filtrate to and through a filter screen 136. In this example, the screen includes apertures ranging size from one to 200 microns. The filter screen 136 therefore captures relatively larger components in the filtrate. Surplus liquid, i.e. cooling agent passes through the filtering surface and is pumped back to the tank. In conventional systems, this surplus liquid is regarded as a waste stream and is discharged. In the present disclosure, it is recognised that there may be still value components entrained in the surplus liquid, and hence returning it to the tank provides a further opportunity for extraction of those value components.

The filtrate may still contain target compounds such as cannabinoids. These compounds can be subsequently removed from the filtrate by additional processing steps (as are discussed in more detail below or as should be known to the skilled addressee).

The use of the separator 132 further processes the filtrate by separating out at least some of the target compounds from the filtrate e.g. the trichomes or relatively large aggregates. It may also serve to filter out at least some of the cooling agent 137 used in the method. This cooling agent passes through the filter screen and may be collected and recycled into one or more of the tanks 102 for re-use for further processing as required. As noted previously, this is an advantage of the present disclosure, since it provides further opportunity to capture any value components, trichomes or fragments of trichomes, missed by the separator, minimises wastage of the cooling agent, and also optimises the processing of the plant material. As shown in FIG. 2, this is via the pipe 124 that is communicative with the shaft 118 of the agitator 106. In doing so, the cooling agent is pumped 139 through a chiller 141 to reduce its temperature.

The remaining filtrate 138, once processed by the separator, flows off the screen 136 and is collected for further processing (140—for example, the clarification and/or dehumidification steps previously referred to). Instead of an angled filter screen, as shown in FIG. 2, in one form the separator may be one or more cyclonic separators. In other forms, the separator may comprise one or more rotary or fixed filters, with a screen of between 1 to 200 microns. Persons skilled in the art will readily utilise and/or adapt known separators suitable for use in the present system.

1.2.6. Operation

The operation of an individual tank of the present disclosure as illustrated in FIG. 2 involves certain key steps. For sake of understanding the operation, and only be way of example, the explanation assumes the tank is dimensioned to have an internal diameter of approximately 2 metres and an internal height of approximately 4 metres, giving an overall operating volume of approximately 4,000 litres. As previously noted, the skilled addressee will readily understand that the tank may be scaled up or down depending on the volume to be processed and therefore may adjust the timing of the operation accordingly.

1.2.6.1. First Step

To begin the process, a batch of the cannabis plant material to be separated out into waste and filtrate is introduced into the tank 102, separately or concurrently with an appropriate amount of cooling agent. The batch is allowed to be fully immersed in the cooling agent, a slurry of chilled water and ice cubes in this example, and have its overall temperature reduced to the point where the trichomes become brittle and ready for separation from the remaining plant material.

In one example, depending on rate of charging of the plant material and chilled water, this first step may take up to 30 minutes.

1.2.6.2. Second Step

The mixture of plant material and chilled water and ice is then vigorously agitated through the use of the agitator 106 and allowed to circulate within the tank 102 and basket 104. This action helps to separate the smaller particulate matter, including the trichomes, from the larger portions of the plant material being processed and into a filtrate.

Throughout this process, the filtrate is allowed to exit the tank 102 via the outlet 108 and be directed to the separator 132. At the separator, the filtrate is further filtered using the angled filter screen 134 to extract at least a portion of the trichomes from same. These trichomes may be entrained in at least some of the chilled water used in the process.

The remainder of the filtrate, which may comprise cooling agent, particulate plant material, suspended solids (including any trichomes not extracted from the filtrate by separator 132) is directed or otherwise recirculated, via pump 139 or gravity feed, back to the tank 102. In conventional systems, this may be considered waste but it has been found that there can still be a significant amount of trichomes present in this filtrate, or able to extracted from the filtrate, to justify its further processing by returning to the tank.

In one example, the mixture is agitated and recirculated for approximately 90 minutes.

1.2.6.3. Third Step

Once the mixture has been agitated and recirculated for the appropriate period of time, the tank 102 can be emptied. This involves the draining of the liquid portion of the mixture, containing the filtrate, through the outlet 108 and to the separator 132.

The residual plant material, the waste from the pre-concentration step, is left behind in the basket 104 which is transferred out of the tank 102. This allows the waste to be removed.

In one example, this step may take up 11 minutes.

1.2.6.4. Fourth Step

To process another batch of harvested cannabis plant material, it is necessary to rinse or otherwise clean the basket and tank with water, together with, or instead of, a cleaning agent such as a mild detergent or solvent. This water is delivered through the agitator 106 (or separate supply if present) and allowed to drain through the outlet 108 of the tank 102, be removed from the system, and then recirculated as a rinsing step as desired.

In one example, the cleaning step may take up to 19 minutes, before the tank 102 is ready to process another batch of harvested cannabis plant material.

In the recited example of the operation of the system, it takes approximately 150 minutes to cycle a single batch of freshly harvested cannabis plant material and the required cooling agent (collectively, approximately a volume of 4,000 litres). Over a 24-hour period, this allows for an hourly processing rate of approximately 1,600 litres through the tank.

5.3 Continuous Processing

A system and method for pre-concentrating harvested cannabis plant material in such a way to achieve continuous processing may involve a plurality of individual systems, each system using a tank, basket and agitator as described herein.

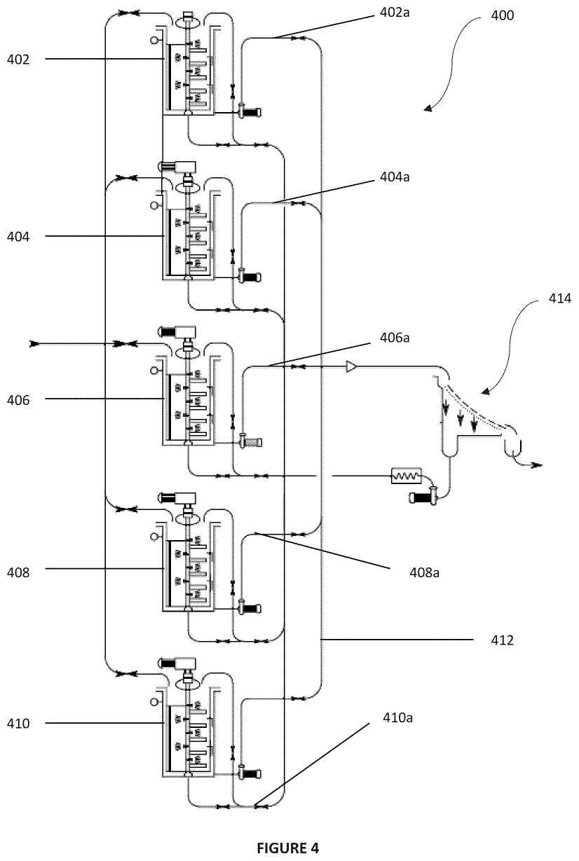

In one example 400, as shown in FIG. 4, there are five individual systems 402, 404, 406, 408 and 410, each with its own agitator and separator. The outlet of each tank may be communicative with a common line 412 leading to a common separator 414.

After the filtrate has been provided to each separator, there is a stream of refined filtrate, i.e. outflow 402a, 404a, 406a, 408a, 410a from each individual system that contains at least some trichomes that has been separated from the filtrate. This outflow is delivered to the common line 412 that is communicative with the single separator 414.

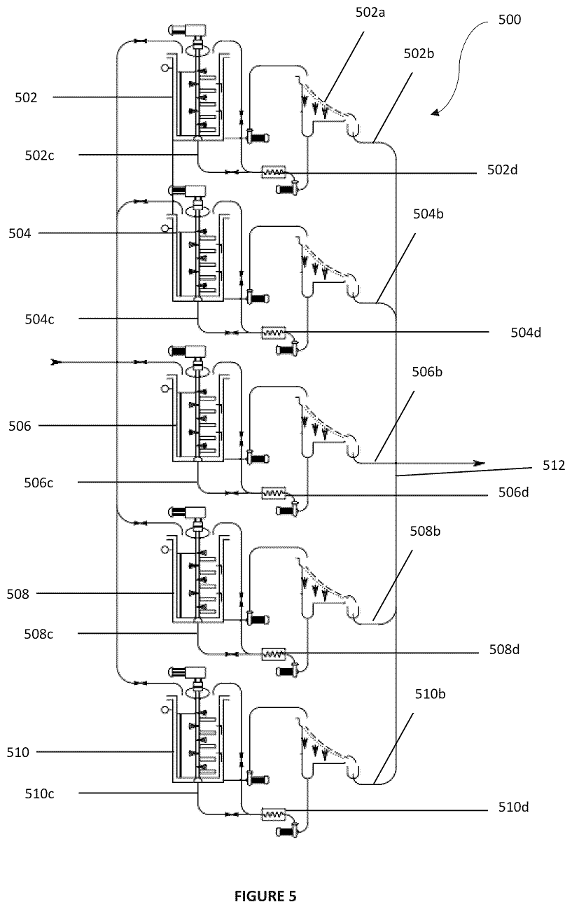

However, in another example of the technology 500, as shown in FIG. 5, each individual system 502, 504, 506, 508, 510 may be provided with its own separator (the first separator —502a, 504a, 506a, 508a, 510a). In this example, the refined filtrate, i.e. outflow 502b, 504b, 506b, 508b, 510b containing the trichomes processed by each first separator is collected and delivered by a common line 512 to a second separator (not shown); this refines the outflow even further to separate out the trichomes before it undergoes further processing. Alternatively, instead of a second separator, the common line may proceed to stations (not shown) for clarification and/or de-watering steps discussed above.

It should be understood the second separator (not shown), if present, receives the combined outflow 502b, 504b, 506b, 508b, 510b from each of the first separators 502a, 504a, 506a, 508a, 510a. The waste stream 502c, 504c, 506c, 508c, 510c from each of the first separators may be recirculated to the originating tank for further processing. In these examples, prior to being returned to the tank, the cooling agent, suspended solids and any trichomes not captured and retained by the first separator may be passed through a chiller 502d, 504d, 506d, 508d, 510d to reduce its temperature.

Optionally, the systems 400, 500 of FIGS. 4 and 5 includes a transfer mechanism (not shown), such as an overhead lifting mechanism (not shown) spanning all tanks in the systems to remove and introduce the agitators and filtering mediums to each tank as required.

The operation of the system 400 of FIG. 4 may be managed and synchronised to ensure there is continuous processing of the freshly harvested cannabis plant material. This should be understood to mean that there is a continuous flow of refined filtrate 402a, 404a, 406a, 408a, 410a to the separator 414. This greatly enhances productivity of the overall pre-concentration step of the processing of freshly harvested cannabis plant material.

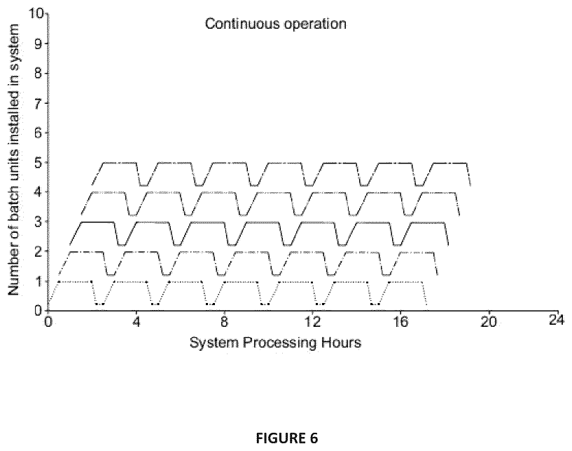

The operation of the system 400 of FIG. 4 may be better understood with reference to FIG. 6, which shows the activity or productivity for the tank of each system 402, 404, 406, 408, 410 as a line. It can be seen that each tank has an initial period of charging the tank with the cooling agent and harvested cannabis plant material (represented by the incline on the activity line for each tank), and then an agitation period (represented by the prolonged flat line on the upper portion of the activity line). It is during the agitation period that there is a supply of filtrate being delivered to the separator 414. At the end of the agitation period, the remaining plant material is removed (the decline on the activity line) and then the tank and filter is rinsed and cleaned (the short flat line on the lower portion of the activity line). During these last two actions (removal of plant material, and then cleaning), the tank is non-productive, with no filtrate being delivered to the separator.

As can be seen, each tank has a start time for its activity that is staggered from the other tanks by at least 30 minutes (corresponding to the length of time taken for the first step in the operation of a single tank). This means that even though each tank of the individual systems 402, 404, 406, 408, 410 has regular periods where it is non-productive, during which there is no agitation taking place, and therefore no filtrate being produced, there is always at least another tank in the overall system 400 still being productive.

The number of individual tanks for optimisation of the entire system 400, i.e. ensuring that at least one tank is always delivering a supply of filtrate to the separator 414, may be determined mathematically:

(Cumulative time of steps 1 to 4)/Time for step 1=No. of tanks

As discussed above in respect of the operation of an individual tank, the first step takes 30 minutes, the second 90 minutes, the third 11 minutes and the fourth 19 minutes, adding to a cumulative total of 150 minutes.

(30 minutes+90 minutes+11 minutes+19 minutes)/30 minutes=5 tanks

This means at a minimum, in this example, five tanks are required to achieve continuous processing; by the time the first tank has finished being cleaned (the fourth step), the fifth tank is just commencing its agitation phase (the second step). It may be useful to have at least one more tank, since this provides some redundancy to the system 400. For example, this could help minimise loss of productivity in the event of equipment failure or maintenance.

In some examples, the same principle may be applied to the agitators, depending on the number of tanks within the system 400. When the first tank has finished its agitation phase (the second step), the agitator may be removed and transferred to a tank ready to commence its agitation phase. This may means that fewer agitators may be required to service the system.

It should be understood that the times provided above for the various steps are by way of example and may vary according to the overall system volume and/or other considerations. For example, larger tanks may require more charge time (the first step) and thus more tanks may be required to ensure that there is a sufficient number to ensure continuous processing is achieved.

It will also be appreciated that this principle can be applied to the system 500 of FIG. 5, in order to optimise flow to the second separator (not shown) or the stations (not shown) forming the clarification and/or de-humidifying steps.

The process optimises resources, both in terms of equipment and staff. For example, once staff have finished charging the first tank in the system with the required amount of harvested cannabis plant material, they can proceed to the next tank and similar charge that while the first tank undergoes its agitation phase. There is no downtime as there would be only a single tank was in operation. In some examples, the system only requires one separator, this receiving the outputs of multiple tanks. As separators may require substantial investment relative to that required for the tanks and agitators, this can mean less capital is required for establishing a facility for processing harvested cannabis plant material.

The present method, system and apparatus is advantageous as it does away with the prolonged drying period that follows harvesting of the cultivated cannabis plants and which is required in conventional techniques. Instead, the plant material may be processed relatively quickly following its harvesting, with minimal drying or dehumidification required as a pre-processing stop. The system may be arranged that multiple batches are processed in a synchronised matter to maximise productivity and potentially achieving greater throughput and efficiency of operation.

Unless the context clearly requires otherwise, through the description and the claims, the words “comprise”, “comprising”, and the like are to be construed in an inclusive sense as opposed to an exclusive or exhaustive sense, that is to say, in the sense of “including, but not limited to”.

Wherein the foregoing description reference has been made to integers or components having known equivalents thereof, those integers are herein incorporated as if individually set forth.

The present disclosure may also be said broadly to consist in the parts, elements, characteristics, and features referred to or indicated in the specification of the application, individually or collectively, in any or all combinations of two or more of said parts, elements, characteristics or features.

Aspects of the present disclosure have been described by way of example only and it should be appreciated that modifications and additions may be made thereto without departing from the scope thereof as defined herein.

It should be noted that various changes and modifications may be made without departing from the spirit and scope of the disclosure as claimed and without diminishing its attendant advantages. It is therefore intended that such changes and modifications be included within the present claims.

Reference to any prior art in this specification is not, and should not be taken as, an acknowledgement or any form of suggestion that the prior art referred to forms part of the common general knowledge in the field of endeavour in any country in the world. The disclosure of any applications, patents and publications cited herein, if any, are herein incorporated by reference.

1. A method of separating trichomes from harvested cannabis plant material, wherein the method comprises the steps of:
a) charging a first vessel with a quantity of harvested cannabis plant material and a quantity of a cooling agent to create a first mixture;
b) agitating the first mixture in the first vessel to produce a first laden quantity of filtrate containing at least some trichomes and a first waste quantity of cannabis plant material;
c) withdrawing the first laden quantity of filtrate from the first vessel and transferring to a separator;
d) operating the separator to remove at least a portion of the trichomes from the first laden quantity of filtrate;
e) charging a second vessel with a quantity of harvested cannabis plant material and a quantity of cooling agent to create a second mixture;
f) agitating the second mixture in the second vessel to produce a second laden quantity of filtrate containing at least some trichomes and a second waste quantity of cannabis plant material;
g) withdrawing the second laden quantity of filtrate from the second vessel and transferring to the separator; and
h) operating the separator to remove at least a portion of trichomes from the second laden quantity of filtrate;
wherein steps e) to g) are timed to provide a substantially continuous outflow of filtrate from the vessels.

2. The method as claimed in claim 1, wherein the method comprises an additional step of:
i) returning at least a portion of the first laden quantity of the filtrate to the first vessel after step d).

3. The method as claimed in claim 2, wherein the method comprises an additional step of:
j) removing the first waste quantity of cannabis plant material from the first vessel during or after step c);
k) cleaning the first vessel; and
l) charging the first vessel with a quantity of harvested cannabis plant material and a quantity of a cooling agent to create a third mixture.

4. The method as claimed in claim 1, wherein the substantially continuous outflow of filtrate from the vessels is to a separator.

5. The method as claimed in claim 1, wherein the substantially continuous outflow of filtrate from the vessels is to a holding tank.

6. The method as claimed in claim 5, wherein the holding tank is communicative with the separator.

7. The method as claimed in claim 1, wherein the cooling agent is a slurry of ice and water and is at a temperature between 0° Celsius and approximately 5° Celsius.

8. The method as claimed in claim 1, wherein the ratio of cooling agent to plant material is from between substantially 0.5:1 to substantially 10:1.

9. The method as claimed in claim 1, wherein steps c) and d) are performed concurrently with step b).

10. The method as claimed in claim 1, wherein step j) comprises cycling a cleaning agent through the vessel.

11. The method as claimed in claim 1, wherein prior to step a), the harvested cannabis plant material undergoes a pre-processing step of being passed through a cutting and/or milling machine.

12. The method as claimed in claim 11, wherein, during the pre-processing step, the harvested cannabis plant material is chilled and/or de-humidified.

13. A system for use in the method of processing harvested cannabis as claimed in claim 1, wherein the system comprises:
a separator; and
a plurality of vessels, wherein each vessel is configured to receive a cooling agent and harvested cannabis plant material and wherein each vessel is provided with:
a charger for harvested cannabis plant material;
a charger for a cooling agent;
an agitator; and
an outlet that is communicative with the separator.

14. The system as claimed in claim 13, wherein each vessel is configured to receive a filtering medium.

15. The system as claimed in claim 14, wherein when the filtering medium is in the vessel, it receives the harvested cannabis plant material.

16. The system as claimed in claim 14, wherein the filtering mediums are substantially cylindrical baskets.

17. The system as claimed in claim 16, wherein the dimensions of the cylindrical baskets are 80% to 95% of the dimensions of the vessel.

18. The system as claimed in claim 16, wherein the cylindrical baskets comprise a continuous side wall with a plurality of perforations.

19. The system as claimed in claim 1, wherein the outlet of each vessel leads to a common line that is communicative with the separator.

20. The system as claimed in claim 1, wherein the system comprises a separator for each vessel.
